(19) United States
(12) Patent Application Publication
(10) Pub. No.: US 2025/0132389 A1
(43) Pub. Date: Apr. 24, 2025

(54) ELECTROLYTE ADDITIVE, ELECTROLYTE, AND LITHIUM ION SECONDARY BATTERY INCLUDING SAME

(71) Applicant: MURATA MANUFACTURING CO., LTD., Kyoto (JP)

(72) Inventors: Fan Yang, Shanghai (CN); Xiaoyin XUE, Shanghai (CN); Cheng ZHU, Shanghai (CN); Kazumasa TAKESHI, Shanghai (CN)

(21) Appl. No.: 18/829,986

(22) Filed: Sep. 10, 2024

(30) Foreign Application Priority Data
Oct. 19, 2023 (CN) 2023113637303

(51) Int. Cl. H01M 10/0567; C07C 305/24; C07C 307/02; C07C 309/76; C07C 311/16; C07C 311/29; C07D 213/71; C07D 233/60; H01M 10/0525

(52) U.S. Cl. CPC H01M 10/0567; C07C 305/24; C07C 307/02; C07C 309/76; C07C 311/16; C07C 311/29; C07D 213/71; C07D 233/60; H01M 10/0525; H01M 2300/004

(57) ABSTRACT

Provided is an electrolyte additive, including a compound of formula (1), wherein R₁ is selected from a substituted or unsubstituted five-membered heterocyclic group, a substituted or unsubstituted six-membered heterocyclic group, or a substituted or unsubstituted amino group; L is a sulfone group, a sulfate ester group, or a sulfonate ester group; and R₂ is selected from a substituted or unsubstituted C₁₋₁₈ alkyl group, a substituted or unsubstituted C₁₋₁₈ alkoxy group, a substituted or unsubstituted conjugated five-membered carbocyclic group or heterocyclic group, a substituted or unsubstituted conjugated six-membered carbocyclic group or heterocyclic group, a di(C₁₋₆ alkyl)amino group, a di(C₆₋₁₂ aryl)amino group, a tri(C₁₋₆ alkyl)silyl group, a substituted or unsubstituted C₃₋₆ alkenyl group, a substituted or unsubstituted C₃₋₆ alkynyl group, or a substituted or unsubstituted ester group. The electrolyte additive, electrolyte, and lithium ion secondary battery comprising same of the present disclosure achieve better protection of electrodes, improve the cycle performance of the battery, and achieve lower post-cycle impedance.

R₁-L-R₂ (1)

### CROSS REFERENCE TO RELATED APPLICATIONS

The present application claims priority to Chinese patent application no. 2023113637303, filed on Oct. 19, 2023, the entire content of which is incorporated herein by reference.

### BACKGROUND

The present disclosure relates to the field of lithium ion secondary batteries, and in particular, to an electrolyte additive, an electrolyte, and a lithium ion secondary battery including same.

In recent years, with the continuous updating of electronic technology, people's demand for battery devices used to support the energy supply of electronic devices has also been increasing. Today, batteries capable of storing more charge and outputting high power are needed. Traditional lead-acid batteries and nickel metal hydride batteries can no longer meet the needs of new electronic products such as mobile devices such as smartphones and fixed devices such as power storage systems. Therefore, lithium batteries have attracted wide attention. In the development of lithium batteries, the capacity and performance thereof have been improved effectively.

In the process of developing an electrolyte for a lithium ion secondary battery, it was found that adding an electrolyte additive would help protect the electrodes of the battery from being corroded by an electrolyte solvent. During the battery formation process (the first charge and discharge cycle), the electrolyte additive will decompose before the electrolyte solvent and forms a film on the surface of the positive or negative electrode, thereby protecting the electrodes. The mechanism of action of the additive is different from that of the solvent in the battery. When the additive forms a film at the positive electrode, it needs to have a higher HOMO energy so that it is easier to lose electrons at the positive electrode and be oxidized to form a SEI film (positive electrode electrolyte interface film). When the additive forms a film at the negative electrode, it needs to have a lower LUMO energy so that it is easier to get electrons at the negative electrode and be reduced to form a SEI film (solid electrolyte interface film). However, the solvent needs to have certain electrochemical stability, and is not oxidized or reduced during charging and discharging of the battery, which would cause failure. In the prior art, different types of electrolyte additives are generally used, so that in the first cycle of the lithium ion secondary battery, different types of electrolyte additives simultaneously form films on the surface of the positive electrode and the negative electrode. However, the method for preparing a lithium ion secondary battery used in the prior art cannot effectively protect electrodes, so that the battery shows a poor cycle retention ratio and a poor impedance after cycling.

Therefore, in order to solve the described problems, there is still a need to develop an electrolyte additive capable of effectively forming an SEI film and a CEI film and ensuring the electrical properties of a lithium ion secondary battery.

### SUMMARY

The present disclosure, in an embodiment, relates to providing a lithium ion secondary battery, and in particular, to an electrolyte additive, an electrolyte, and a lithium ion secondary battery including same.

In an embodiment of the present disclosure, provided is an electrolyte additive, comprising a compound of formula (1):

R₁-L-R₂ (1)

wherein R₁ is selected from a substituted or unsubstituted five-membered heterocyclic group, a substituted or unsubstituted six-membered heterocyclic group, or a substituted or unsubstituted amino group; L is a sulfone group, a sulfate ester group, or a sulfonate ester group; and R₂ is selected from a substituted or unsubstituted C₁₋₁₈ alkyl group, a substituted or unsubstituted C₁₋₁₈ alkoxy group, a substituted or unsubstituted conjugated five-membered carbocyclic group or heterocyclic group, a substituted or unsubstituted conjugated six-membered carbocyclic group or heterocyclic group, a di(C₁₋₆ alkyl)amino group, a di(C₆₋₁₂ aryl)amino group, a tri(C₁₋₆ alkyl)silyl group, a substituted or unsubstituted C₃₋₆ alkenyl group, a substituted or unsubstituted C₃₋₆ alkynyl group, an ether group, or a substituted or unsubstituted ester group.

Further, in the electrolyte additive, in an embodiment, in the compound of formula (1), R₁ is a substituted or unsubstituted pyridyl group, a substituted or unsubstituted pyrazinyl group, a substituted or unsubstituted imidazolyl group, or a (C₁₋₃ alkyl) substituted or unsubstituted amino group.

Further, in the electrolyte additive, in an embodiment, in the compound of formula (1), R₁ is a pyridyl group, a pyrazinyl group, an imidazolyl group, an amino group, a dimethylamino group, or a diethylamino group.

Further, in the electrolyte additive, in an embodiment, in the compound of formula (1), R₂ is selected from a substituted or unsubstituted C₁₋₈ alkyl group, a substituted or unsubstituted C₁₋₈ alkoxy group, a substituted or unsubstituted conjugated six-membered carbocyclic group or heterocyclic group, a substituted or unsubstituted —COO(C₁₋₆), di(C₁₋₃ alkyl)amino or di(C₆₋₁₂ aryl)amino group, a cyano group, or an ether group.

Further, in the electrolyte additive, in an embodiment, in the compound of formula (1), R₂ is selected from a C₁₋₃ alkyl substituted or unsubstituted conjugated six-membered carbocyclic group or heterocyclic group, a C₁₋₃ alkoxy substituted conjugated six-membered carbocyclic group or heterocyclic group, a fluoro substituted conjugated six-membered carbocyclic group or heterocyclic group, a fluoromethyl substituted conjugated six-membered carbocyclic group or heterocyclic group, a difluoromethyl substituted conjugated six-membered carbocyclic group or heterocyclic group, a trifluoromethyl substituted conjugated six-membered carbocyclic group or heterocyclic group, a cyano substituted conjugated six-membered carbocyclic group or heterocyclic group, a cyano substituted or unsubstituted C₁₋₈ alkyl group, a cyano substituted or unsubstituted C₁₋₈ alkoxy group, a di(C₁₋₃ alkyl)amino group, a di(C₆₋₁₂ aryl)amino group, and a cyano group.

Further, in the electrolyte additive, in an embodiment, in the compound of formula (1), R₂ is selected from phenyl, p-methylphenyl, p-ethylphenyl, m-methylphenyl, m-ethylphenyl, 3, 5-dimethylphenyl, 3, 5-diethylphenyl, o-methylphenyl, 2, 6-dimethylphenyl, imidazolyl, pyridyl, pyrimidyl, pyridazinyl, pyrazinyl, —CH₃, —OCH₃, —C₂H₅, —OC₂H₅, —C₃H₇, —OC₃H₇, n-butyl, isobutyl, t-butyl, —C₆H₁₃, —C₁₈H₃₇, —COOCH₃, —COOC₂H₅, —CN, —CH₂CN, dimethylamino, diethylamino, dipropylamino, trimethylsilyl, triethylsilyl, triisopropylsilyl, vinyl, or ethynyl.

Further, in the electrolyte additive, in an embodiment, the compound of formula (1) is selected from:

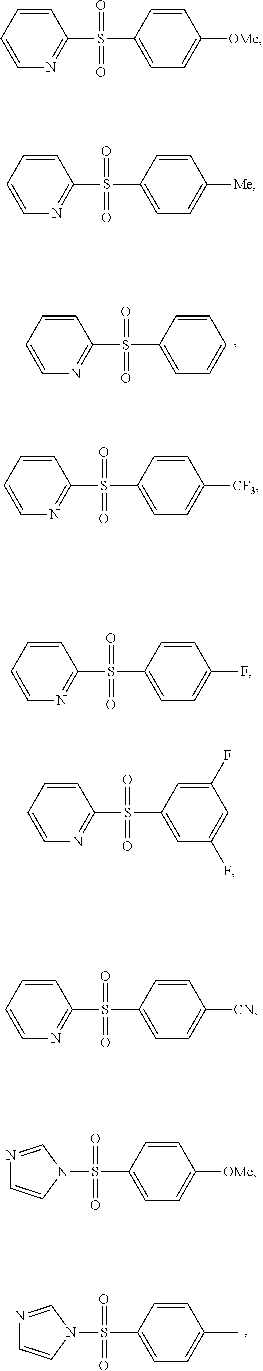

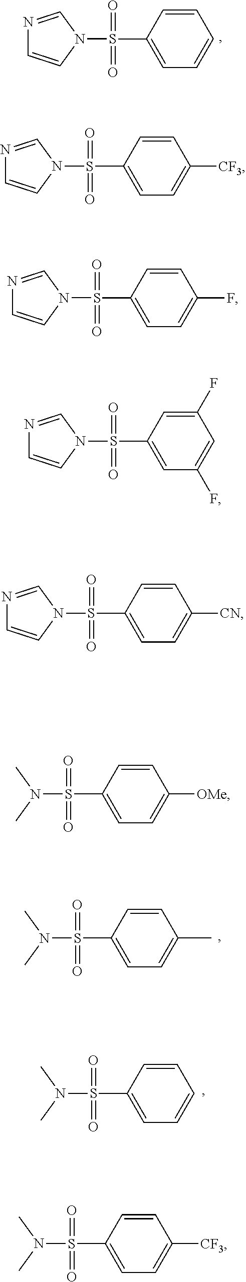

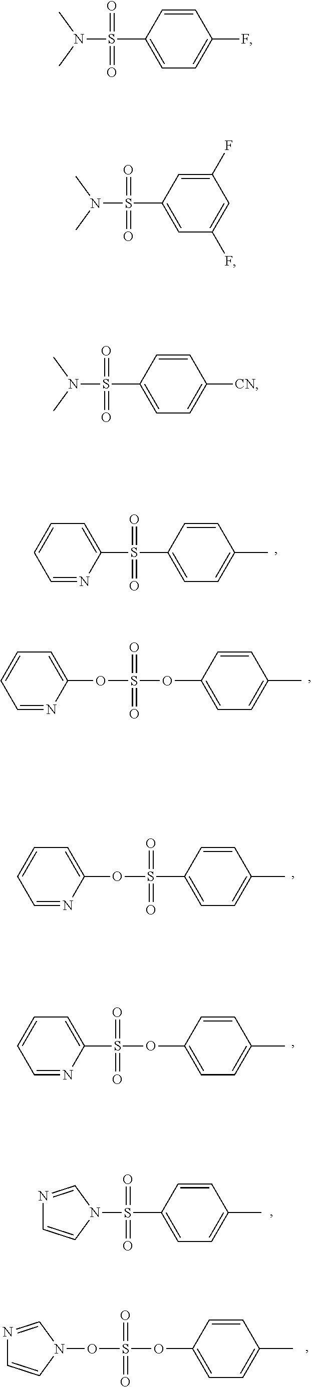

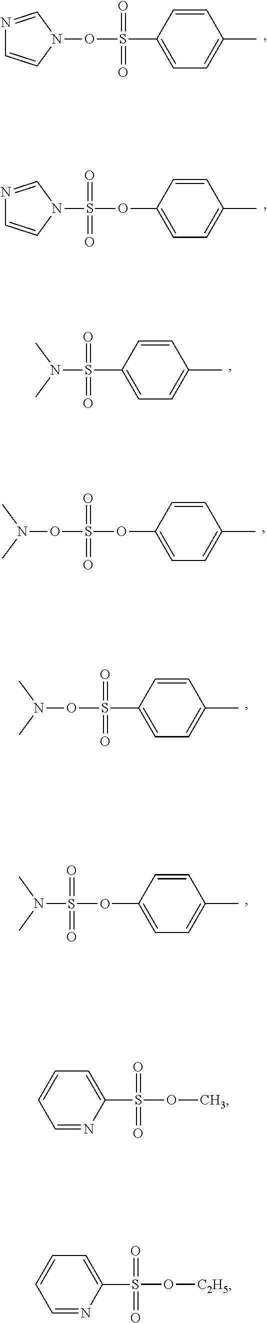

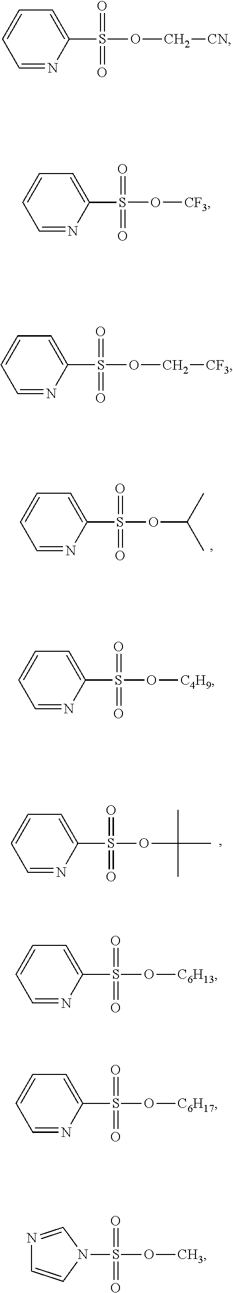

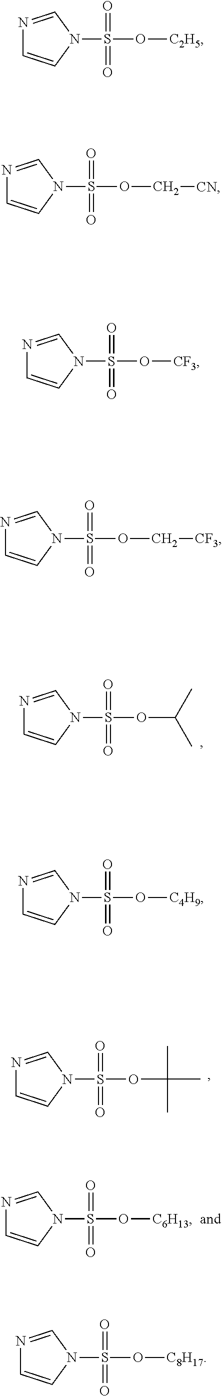

According to another embodiment of the present disclosure, in an embodiment, an electrolyte is provided and includes organic solvent, lithium salt, and the electrolyte additive described above

Further, in the electrolyte, in an embodiment, the amount of the electrolyte additive is in the range of 0.01 parts by weight to 0.25 parts by weight based on 100 parts by weight of the total weight of the organic solvent and the lithium salt; and preferably, the amount of the electrolyte additive is in the range of 0.1 parts by weight to 0.2 parts by weight.

Further, in the electrolyte, in an embodiment, the lithium salt is selected from LiCl, LiBr, LiPF₆, LiBF₄, LiAsF₆, LiClO₄, LiB(C₆H₅)₄, LiCH₃SO₃, LiCF₃SO₃, LiN(SO₂F)₂, LiN(SO₂CF₃)₂, LiC(SO₂CF₃)₃, LiAlCl₄, LiSiF₆, or any combinations thereof.

Further, in the electrolyte, in an embodiment, the organic solvent is selected from ethylene carbonate, propylene carbonate, butylene carbonate, fluoroethylene carbonate, diethyl carbonate, dipropyl carbonate, methyl ethyl carbonate, vinyl carbonate, dimethyl carbonate, or any combinations thereof.

According to yet another embodiment of the present disclosure, a lithium ion secondary battery is provided and includes a positive electrode sheet, a negative electrode sheet, a separator, and the electrolyte of the present disclosure.

The electrolyte additive, the electrolyte, and the lithium ion secondary battery including same of the present disclosure provide better protection of electrodes, improve the cycle performance of the battery, and provide a lower post-cycle impedance according to an embodiment.

### DETAILED DESCRIPTION

The present disclosure is described below in further detail according to an embodiment. It is important to note that the embodiments of the present disclosure and the characteristics in the embodiments can be combined under the condition of no conflicts. Hereinafter, the present disclosure will be described in detail with reference to the embodiments. The following embodiments are merely exemplary, and do not limit the scope of protection of the present disclosure.

As explained in the background art, in the related art, an electrolyte additive added to a lithium ion secondary battery cannot effectively protect the positive electrode and the negative electrode of the battery, and disadvantageously deteriorates the electrical properties of the lithium ion secondary battery. In an embodiment of the present disclosure, an electrolyte additive is provided and includes a compound of formula (1):

R₁-L-R₂ (1)

wherein R₁ is selected from a substituted or unsubstituted five-membered heterocyclic group, a substituted or unsubstituted six-membered heterocyclic group, or a substituted or unsubstituted amino group; L is a sulfone group, a sulfate ester group, or a sulfonate ester group; and R₂ is selected from a substituted or unsubstituted C₁₋₁₈ alkyl group, a substituted or unsubstituted C₁₋₁₈ alkoxy group, a substituted or unsubstituted conjugated five-membered carbocyclic group or heterocyclic group, a substituted or unsubstituted conjugated six-membered carbocyclic group or heterocyclic group, a di(C₁₋₆ alkyl)amino group, a di(C₆₋₁₂ aryl)amino group, a tri(C₁₋₆ alkyl)silyl group, a substituted or unsubstituted C₃₋₆ alkenyl group, a cyano group, a substituted or unsubstituted C₃₋₆ alkynyl group, an ether group, or a substituted or unsubstituted ester group.

In an embodiment, the electrolyte additive of the present disclosure can simultaneously undergo a redox reaction on the positive electrode and the negative electrode of a battery, and products are deposited on the positive electrode and the negative electrode, thereby simultaneously forming an SEI film and a CEI film on the surface thereof.

The electrolyte additive of the present disclosure, in an embodiment, is as represented by formula (1) above, wherein R₁ has a Lewis basic structure. R₁ diffuses the electron cloud distribution of the HOMO through bridging of a sulfone group (or a sulfate ester group or a sulfonate ester group) having two oxygen-containing double bonds. At the R₂ position, an electron withdrawing structure or a π-conjugate structure is used to further diffuse the electron cloud, thereby inhibiting the basicity of R₁, and further inhibiting the Lewis basicity of the whole electrolyte additive molecule. Moreover, the alkaline property of the whole molecule can be finely regulated by using the substituent at R₂, so as to adjust the film-forming properties thereof in the battery. When the battery including the electrolyte additive of the present disclosure is charged for the first time, when the positive electrode is delithiated, a local electron deficient region is formed at the delithiated position, and the alkaline portion rich in electrons in the compound of formula (1) will react on the surface of the positive electrode, so as to ensure that the additive forms a CEI film on the positive electrode. In an embodiment, after the battery using the electrolyte additive of the present disclosure is assembled, a CEI film is formed on the electrode before the battery is charged and discharged for the first time due to a local infiltration reaction and the like after the battery is assembled, so that the open circuit voltage of the battery changes. In addition, in the compound of formula (1) of the present disclosure, oxidation and reduction reactions occur simultaneously on the positive electrode and the negative electrode, so that on the one hand, an SEI film and a CEI film of a low-impedance component are formed on the positive electrode and the negative electrode, and the battery impedance is reduced, and on the other hand, the dissolution of a transition metal can be inhibited, thereby improving the cycle performance of the battery.

In an embodiment, in the compound of formula (1), R₁ is a substituted or unsubstituted pyridyl group, a substituted or unsubstituted pyrazinyl group, a substituted or unsubstituted imidazolyl group, or a (C₁₋₃ alkyl) substituted or unsubstituted amino group. As the compound of formula (1) contains N atom, after decomposition products are deposited on the positive electrode and the negative electrode, the N atom contained therein can interact with free lithium ions in the electrolyte, thereby improving the intercalation and deintercalation of the lithium ions related to the electrodes, and improving the transmission efficiency of the lithium ions.

In an embodiment of the present disclosure, in the compound of formula (1), R₁ is a pyridyl group, a pyrazinyl group, an imidazolyl group, an amino group, a dimethylamino group, or a diethylamino group.

In an embodiment, in the compound of formula (1) of the present disclosure, R₂ is selected from a substituted or unsubstituted C₁₋₈ alkyl group, a substituted or unsubstituted C₁₋₈ alkoxy group, a substituted or unsubstituted conjugated six-membered carbocyclic group or heterocyclic group, a substituted or unsubstituted —COO(C₁₋₆), di(C₁₋₃ alkyl)amino or di(C₆₋₁₂ aryl)amino group, a cyano group, or an ether group.

In an embodiment, in the compound of formula (1) of the present disclosure, R₂ is selected from a C₁₋₃ alkyl substituted or unsubstituted conjugated six-membered carbocyclic group or heterocyclic group, a C₁₋₃ alkoxy substituted conjugated six-membered carbocyclic group or heterocyclic group, a fluoro substituted conjugated six-membered carbocyclic group or heterocyclic group, a fluoromethyl substituted conjugated six-membered carbocyclic group or heterocyclic group, a difluoromethyl substituted conjugated six-membered carbocyclic group or heterocyclic group, a trifluoromethyl substituted conjugated six-membered carbocyclic group or heterocyclic group, a cyano substituted conjugated six-membered carbocyclic group or heterocyclic group, a cyano substituted or unsubstituted C₁₋₈alkyl group, a cyano substituted or unsubstituted C₁₋₈alkoxy group, a di(C₁₋₃ alkyl)amino or di(C₆₋₁₂ aryl)amino group, and a cyano group.

In an embodiment, in the compound of formula (1), R₂ is selected from phenyl, p-methylphenyl, p-ethylphenyl, m-methylphenyl, m-ethylphenyl, 3,5-dimethylphenyl, 3,5-diethylphenyl, o-methylphenyl, 2,6-dimethylphenyl, imidazolyl, pyridyl, pyrimidinyl, pyridazinyl, pyrazinyl, —CH₃, —OCH₃, —C₂H₅, —OC₂H₅, —C₃H₇, —OC₃H₇, n-butyl, isobutyl, t-butyl, —C₆H₁₃, —C₁₈H₃₇, —COOCH₃, —COOC₂H₅, —CN, —CH₂CN, dimethylamino, diethylamino, dipropylamino, trimethylsilyl, triethylsilyl, triisopropylsilyl, vinyl, or ethynyl.

In an embodiment of the present disclosure, the chemical formula of formula (1) can be selected from:

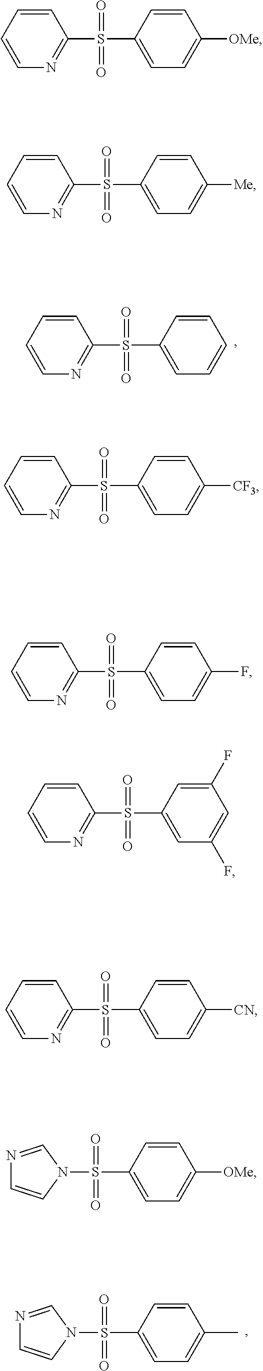

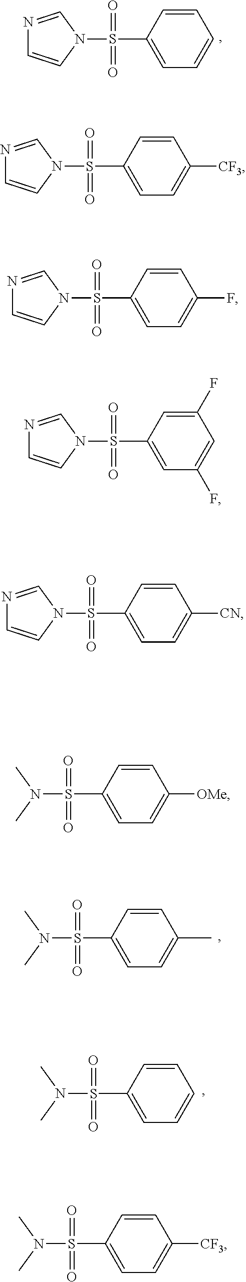

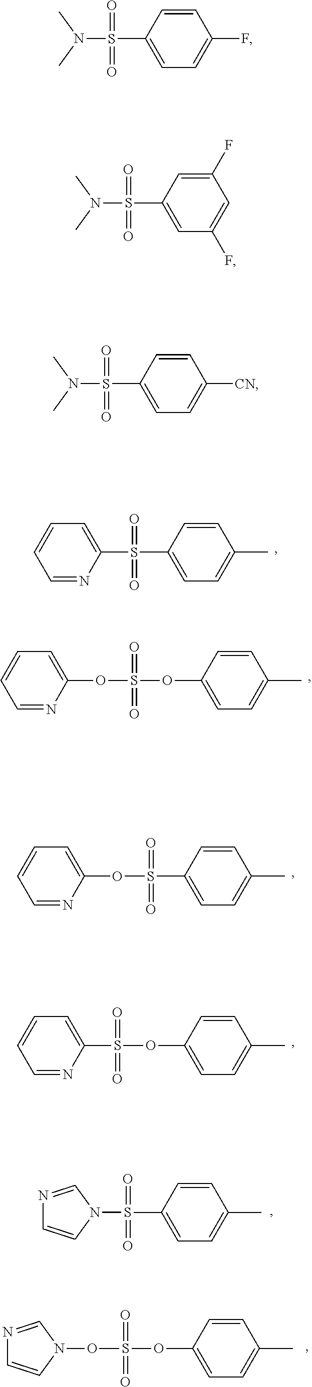

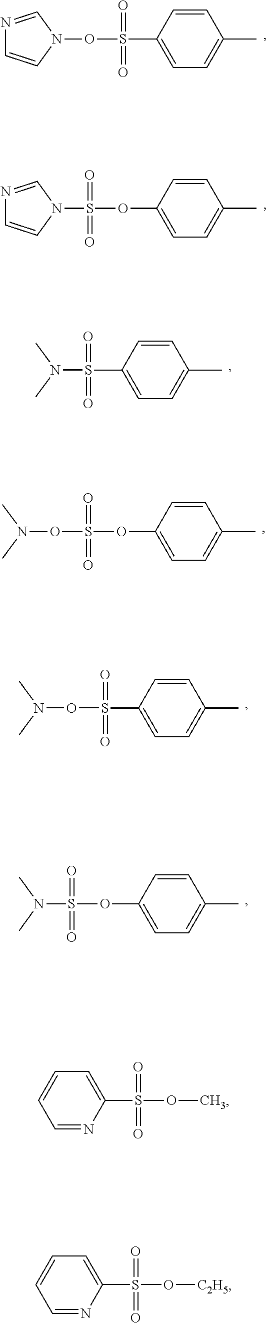

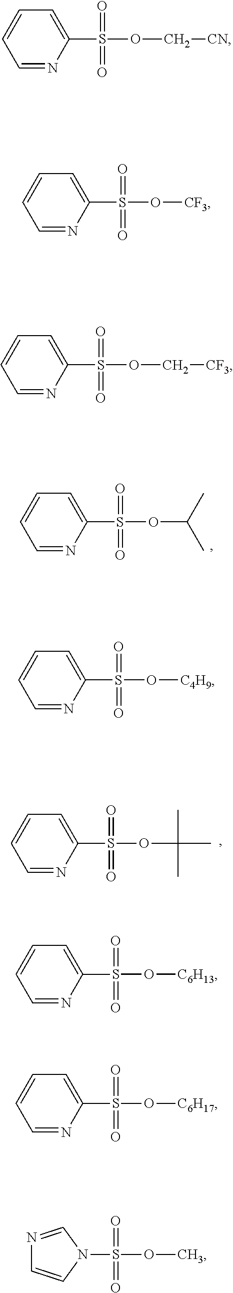

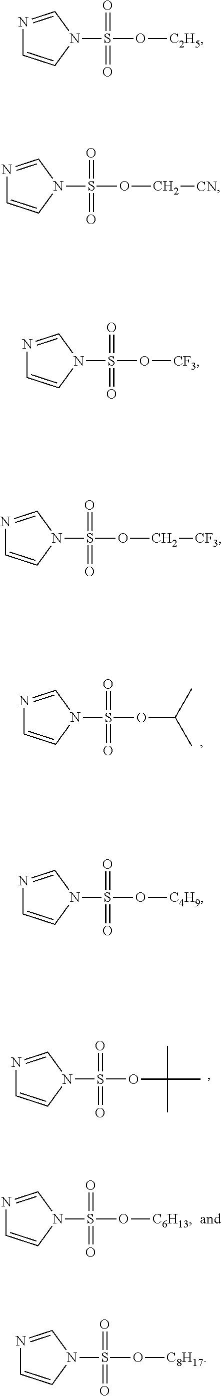

In an embodiment of the present disclosure, provided is an electrolyte comprising an organic solvent, a lithium salt, and the electrolyte additive as described hereinabove. Due to the inclusion of the electrolyte additive of the present disclosure, the electrolyte of the present disclosure can more effectively form a CEI film on the surface of the positive electrode and form an SEI film on the surface of the negative electrode during the first cycle of the battery, thereby suppressing decomposition of the solvent and corrosion of the electrodes. In addition, when the battery comprising the electrolyte additive of the present disclosure is charged for the first time, when the positive electrode is delithiated, a local electron deficient region is formed at the delithiated position, and the alkaline portion rich in electrons in the compound of formula (1) will react on the surface of the positive electrode, so as to ensure that the additive forms a CEI film on the positive electrode. Further, after the battery using the electrolyte additive of the present disclosure is assembled, a CEI film is formed on the electrode before the battery is charged and discharged for the first time due to a local infiltration reaction and the like after the battery is assembled.

In an embodiment of the present disclosure, in the electrolyte of the present disclosure, the amount of the electrolyte additive is in the range of 0.01 parts by weight to 0.25 parts by weight based on 100 parts by weight of the total weight of the organic solvent and the lithium salt; and preferably, the amount of the electrolyte additive is in the range of 0.1 parts by weight to 0.2 parts by weight. Within the described ranges in parts by weight, the electrolyte additive can be rapidly decomposed in the first cycle of the battery or in the infiltration process before the first charge, and can be attached to the positive electrode and the negative electrode to form the CEI film and the SEI film. In the case where only the electrolyte additive of the present disclosure is used, if the amount of the electrolyte additive is less than 0.01 parts by weight, the formed SEI film and CEI film cannot be completely attached to the positive electrode and the negative electrode, and during the subsequent use, the solvent will adversely corrode the electrodes and affect the electrical properties of the battery, for example, will increase the resistance of the battery, reduce the cycle retention ratio, etc. If the amount of the electrolyte additive is higher than 0.25 parts by weight, the added electrolyte additive cannot be completely decomposed and remains in the electrolyte or is directly attached to the electrodes without being decomposed, so that the impedance of the electrolyte or the surface of the electrodes is increased, thereby disadvantageously reducing the electrical properties of the battery.

In an embodiment of the present disclosure, the minimum value of the amount of the electrolyte additive contained in the electrolyte depends on various combinations of the lithium salt and the organic solvent, and based on 100 parts by weight of the total weight of the organic solvent and the lithium salt, should be greater than 0.01 parts by weight, 0.02 parts by weight, 0.03 parts by weight, 0.04 parts by weight, 0.05 parts by weight, 0.06 parts by weight, 0.07 parts by weight, 0.08 parts by weight, 0.09 parts by weight, 0.1 parts by weight, 0.11 parts by weight, 0.12 parts by weight, 0.13 parts by weight, 0.14 parts by weight, 0.15 parts by weight, 0.16 parts by weight, 0.17 parts by weight, 0.18 parts by weight, 0.19 parts by weight, or 0.2 parts by weight. In addition, depending on the combination of the organic solvent and the lithium salt, based on 100 parts by weight of the total weight of the organic solvent and the lithium salt, the maximum value of the amount of the electrolyte additive contained in the electrolyte should be less than 0.25 parts by weight, 0.24 parts by weight, 0.23 parts by weight, 0.22 parts by weight, 0.21 parts by weight, 0.2 parts by weight, 0.19 parts by weight, 0.18 parts by weight, 0.17 parts by weight, 0.16 parts by weight, 0.15 parts by weight, 0.14 parts by weight, 0.13 parts by weight, 0.12 parts by weight, 0.11 parts by weight, or 0.1 parts by weight.

Specifically, based on 100 parts by weight of the total weight of the organic solvent, the lithium salt, and the film-forming additive, the amount of the electrolyte additive in the electrolyte may be in the range of 0.01 parts by weight to 0.25 parts by weight, 0.02 parts by weight to 0.24 parts by weight, 0.03 parts by weight to 0.23 parts by weight, 0.04 parts by weight to 0.22 parts by weight, 0.05 parts by weight to 0.21 parts by weight, 0.06 parts by weight to 0.2 parts by weight, 0.07 parts by weight to 0.19 parts by weight, 0.08 parts by weight to 0.18 parts by weight, 0.09 parts by weight to 0.17 parts by weight, 0.1 parts by weight to 0.16 parts by weight, 0.01 parts by weight to 0.24 parts by weight, 0.01 parts by weight to 0.23 parts by weight, 0.01 parts by weight to 0.22 parts by weight, 0.01 parts by weight to 0.21 parts by weight, 0.01 parts by weight to 0.2 parts by weight, 0.02 parts by weight to 0.25 parts by weight, 0.03 parts by weight to 0.25 parts by weight, 0.04 parts by weight to 0.25 parts by weight, 0.05 parts by weight to 0.25 parts by weight, 0.06 parts by weight to 0.25 parts by weight, 0.07 parts by weight to 0.25 parts by weight, 0.08 parts by weight to 0.25 parts by weight, 0.09 parts by weight to 0.25 parts by weight, 0.1 parts by weight to 0.25 parts by weight, or 0.1 parts by weight to 0.2 parts by weight.

In the present disclosure, the film-forming additive which can be used is selected from fluoroethylene carbonate (FEC), vinylene carbonate, halogenated cyclic carbonates, sulfonates, anhydrides, polynitrile compounds, diisocyanate compounds, phosphates, borates, or any combinations thereof. These film-forming additives may be used alone. In an embodiment of the present disclosure, the preferred film-forming additive is fluoroethylene carbonate.

The present disclosure has no particular limitation on the lithium salt component contained in the electrolyte, and any electrolyte known in the prior art that can be used for lithium batteries can be used. Examples of the lithium salt include, but are not limited to, LiCl, LiBr, LiPF₆, LiBF₄, LiAsF₆, LiClO₄, LiB(C₆H₅)₄, LiCH₃SO₃, LiCF₃SO₃, LiN(SO₂F)₂, LiN(SO₂CF₃)₂, LiC(SO₂CF₃)₃, LiAlCl₄, LiSiF₆, or any combinations thereof.

In the present disclosure, the organic solvent used in the electrolyte may be any non-aqueous solvent heretofore used for a non-aqueous electrolyte. Examples include, but are not limited to, linear or cyclic carbonates such as ethylene carbonate, propylene carbonate, butylene carbonate, diethyl carbonate, dimethyl carbonate, methyl ethyl carbonate, dipropyl carbonate, and fluoroethylene carbonate; ethers such as 1, 2-dimethoxyethane, 1, 2-diethoxyethane, γ-butyrolactone, tetrahydrofuran, 2-methyltetrahydrofuran, 1, 3-dioxolane, 4-methyl-1, 3-dioxolane, and diethyl ether; sulfones such as sulfolane and methylsulfolane; nitriles such as acetonitrile, propionitrile, and acrylonitrile; esters such as acetates, propionates, and butyrates. These non-aqueous solvents may be used alone or a plurality of solvents may be used in combination. In an embodiment of the present disclosure, preferred electrolytes include ethylene carbonate, propylene carbonate, butylene carbonate, fluoroethylene carbonate, diethyl carbonate, dipropyl carbonate, methyl ethyl carbonate, vinyl carbonate, dimethyl carbonate, or any combinations thereof. In a preferred embodiment, at least one carbonate is used as the organic solvent of the electrolyte of the present disclosure. In other preferred embodiments, the described non-aqueous solvents may be used in any combination to form an electrolyte complying with specific requirements.

In an embodiment of the present disclosure, provided is a lithium ion secondary battery, comprising: a positive electrode sheet, a negative electrode sheet, a separator, and the described electrolyte. As the lithium ion secondary battery of the present disclosure uses the described electrolyte, it has excellent cycle performance and reduced impedance.

The positive electrode sheet of the present disclosure includes a positive electrode current collector and a positive electrode active material layer containing a positive electrode active material. A positive electrode active material layer is formed on both surfaces of the positive electrode current collector. As the positive electrode current collector, a metal foil such as an aluminum foil, a nickel foil, and a stainless steel foil may be used.

The positive electrode active material layer includes, as a positive electrode active material, one or two or more of positive electrode materials capable of absorbing and releasing lithium ions, and may contain, as necessary, another material such as a positive electrode binder and/or a positive electrode conductive agent.

Preferably, the positive electrode material is a lithium-containing compound. Examples of such lithium-containing compound include a lithium-transition metal composite oxide, a lithium-transition metal phosphate compound, and the like. The lithium-transition metal composite oxide is an oxide containing Li and one or two or more transition metal elements as constituent elements, and the lithium-transition metal phosphate compound is a phosphate compound containing Li and one or two or more transition metal elements as constituent elements. Among them, the transition metal element is favorably any one or two or more of Co, Ni, Mn, Fe, and the like.

Examples of the lithium-transition metal composite oxide include, for example, LiCoO₂, LiNiO₂, and the like. Examples of the lithium-transition metal phosphate compound include, for example, LiFePO₄, LiFe₁₋ᵤMnᵤPO₄ (0<u<1), and the like.

In an embodiment of the present disclosure, the positive electrode material may be a ternary positive electrode material such as nickel cobalt lithium aluminate (NCA) or nickel cobalt lithium manganate (NCM). Specific examples may be NCA, LixNiyCozAl1−y−zO₂ (1≤x≤1.2, 0.5≤y≤1, and 0≤z≤0.5); and NCM, LiNixCoyMnzO₂ (x+y+z=1, 0<x<1, 0<y<1, and 0<z<1). Specific examples of the positive electrode material may include, but are not limited to, LiNiO₂, LiCoO₂, LiCo_{0.98}Al_{0.01}Mg_{0.01}O₂, LiNi_{0.5}Co_{0.2}Mn_{0.3}O₂, LiNi_{0.8}Co_{0.15}Al_{0.05}O₂, LiNi_{0.33}Co_{0.33}Mn_{0.33}O₂, Li_{1.2}Mn_{0.52}Co_{0.175}Ni_{0.1}O₂, and Li_{1.15}(Mn_{0.65}Ni_{0.22}Co_{0.13})O₂, LiFePO₄, LiMnPO₄, LiFe_{0.5}Mn_{0.5}PO₄, and LiFe_{0.3}Mn_{0.7}PO₄.

Further, the positive electrode material may be, for example, any one or two or more of oxides, disulfides, chalcogenides, conductive polymers, and the like. Examples of the oxides include, for example, titanium oxide, vanadium oxide, manganese dioxide, and the like. Examples of the disulfide includes, for example, titanium disulfide, molybdenum sulfide, and the like. Examples of the chalcogenides include, for example, niobium selenide and the like. Examples of the conductive polymers include, for example, sulfur, polyaniline, polythiophene, and the like. However, the positive electrode material may be a different material from those described above.

Examples of the positive electrode conductive agent include carbon materials such as graphite, carbon black, acetylene black, and Ketjen black. These may be used singly, or two or more thereof may be used in mixture. It is to be noted that the positive electrode conductive agent may be a metal material, a conductive polymer, or the like as long as it has electrical conductivity.

Examples of the positive electrode binder include, for example, synthetic rubber and polymer materials. The synthetic rubber may be, for example, styrene butadiene rubber, fluororubber, and ethylene propylene diene. The polymer material may be, for example, polyvinylidene fluoride, polyvinyl alcohol, carboxymethyl cellulose (CMC), starch, hydroxypropyl cellulose, regenerated cellulose, polyvinyl pyrrolidone, tetrafluoroethylene, polyethylene, polypropylene, lithium polyacrylate, ethylene-propylene-diene terpolymer (EPDM), sulfonated EPDM, and polyimide. These may be used singly, or two or more thereof may be used in mixture.

The negative electrode sheet of the present disclosure comprises a negative electrode current collector and a negative electrode active material layer containing a negative electrode active material. The negative electrode active material layer is formed on both surfaces of the negative electrode current collector. As the negative electrode current collector, a metal foil such as a copper foil, a nickel foil, and a stainless steel foil may be used.

The negative electrode active material layer comprises, as a negative electrode active material, a material capable of absorbing and releasing lithium ions, and may comprise, as necessary, another material such as a negative electrode binder and/or a negative electrode conductive agent. Details of the negative electrode binder and the negative electrode conductive agent are the same as those of the positive electrode binder and the negative electrode conductive agent, for example.

The active material of the negative electrode is any one or a combination of more selected from lithium metal, lithium alloys, carbon materials, silicon or tin, and oxides thereof.

As a carbon material has a low potential when absorbing lithium ions, a high energy density can be obtained, and the battery capacity can be increased. In addition, the carbon material also functions as a conductive agent. Such carbon material is, for example, natural graphite, artificial graphite, a material obtained by coating them on amorphous carbon, or the like. Note that the shape of the carbon material is fibrous, spherical, granular, scale-like, or the like. The silicon-based material includes nano silicon, silicon alloys, and silicon-carbon composite materials formed by compounding SiO_{w} and graphite. Preferably, SiO_{w} is silicon monoxide, silicon oxide, or other silicon-based material.

Otherwise, the negative electrode material may be, for example, one or two or more of easily graphitizable carbon, hardly graphitizable carbon, metal oxides, polymer compounds, and the like. Examples of the metal oxides include, for example, iron oxide, ruthenium oxide, molybdenum oxide, and the like. Examples of the polymer compounds include, for example, polyacetylene, polyaniline, polypyrrole, and the like. However, the negative electrode material may be another material different from those described above.

The separator of the present disclosure is used to separate the positive electrode sheet and the negative electrode sheet in the battery and allow ions to pass therethrough while preventing current short-circuiting due to contact between the two electrode sheets. The separator is, for example, a porous film formed of a synthetic resin, ceramic, or the like, and may be a laminated film in which two or more porous films are laminated. Examples of the synthetic resin include, for example, polytetrafluoroethylene, polypropylene, polyethylene, cellulose, and the like.

In an embodiment of the present disclosure, during charging, for example, lithium ions are released from the positive electrode and are absorbed in the cathode through the non-aqueous electrolyte impregnated in the separator. During discharging, for example, lithium ions are released from the negative electrode and are absorbed in the anode through the non-aqueous electrolyte impregnated in the separator.

The present disclosure will be further described in detail according to an embodiment, which should not be construed as limiting the scope of protection of the present disclosure.

### Example 1

### Preparation of Electrolyte

20.8 g of ethylene carbonate, 56.7 g of dimethyl carbonate, 3.5 g of ethyl methyl carbonate, 0.4 g of lithium tetrafluoroborate, and 18.6 g of lithium hexafluorophosphate were mixed to prepare a basic electrolyte. 0.01 g of an electrolyte additive is added to the described electrolyte, and stirred until uniform so as to obtain an electrolyte for use. The electrolyte additive used was as represented by the following formula:

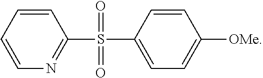 [SMILES: COc1ccc(S(=O)(=O)c2ccccn2)cc1]

### Preparation of Positive Electrode

95.5 g of a positive electrode active material lithium nickel cobalt aluminate, 2.5 g of conductive carbon black, 1.9 g of polyvinylidene fluoride, and 0.1 g of a dispersant were mixed to obtain a positive electrode mixture, and the obtained mixture was dispersed in N-methylpyrrolidone to obtain a positive electrode mixture slurry. Subsequently, the obtained positive electrode mixture slurry was uniformly coated onto an aluminum foil to obtain a positive electrode current collector, the positive electrode current collector was dried, and a positive electrode sheet was formed by using a punch forming process.

### Preparation of Negative Electrode

95.85 g of a mixture of silicon monooxide (SiOₓ, 1<x<2) and graphite powder (mass ratio of 1:11), 1 g of Super P, 3.15 g of a binder CMC (sodium carboxymethyl cellulose) and SBR (styrene butadiene rubber) (wherein the mass ratio of CMC to SBR was 1:1), and an appropriate amount of water were stirred to prepare a negative electrode slurry. The obtained negative electrode slurry was then uniformly coated on a copper foil to obtain a negative electrode current collector, the negative electrode current collector was dried, and a negative electrode sheet was formed by a punch forming process.

### Assembly of Battery

A CR2016 button cell was assembled in a dry laboratory. The positive electrode sheet prepared in the described steps was used as the positive electrode, the negative electrode sheet was used as the negative electrode, and the electrolyte prepared in the described steps was used as the electrolyte of a battery. The positive electrode, the negative electrode, the separator, and a battery housing for the button cell were assembled. After the battery was assembled, the battery was allowed to stand for 24 hours and aged, thereby obtaining a button battery.

### Examples 2-60

The batteries of Examples 2-60 and Comparative Examples 1-5 were prepared by the same method as in Example 1, and for the differences, see Table 1 below.

TABLE 1

| | | Additive amount of electrolyte |
|---|---|---|
| | Type of electrolyte additive | additive |
| Example 1 Example 2 Example 3 Example 4 Example 5 | 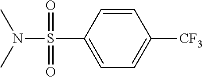 [SMILES: CN(C)S(=O)(=O)c1ccc(C(F)(F)F)cc1] | 0.01 g 0.05 g 0.10 g 0.12 g 0.15 g |
| Example 6 | | 0.18 g |
| Example 7 | | 0.20 g |
| Example 8 | | 0.25 g |
| Example 9 | 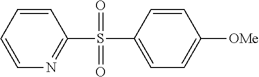 [SMILES: COc1ccc(S(=O)(=O)c2ccccn2)cc1] | 0.10 g |
| Example 10 | 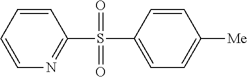 [SMILES: Cc1ccc(S(=O)(=O)c2ccccn2)cc1] | 0.10 g |
| Example 11 | 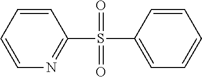 [SMILES: O=S(=O)(c1ccccc1)c1ccccn1] | 0.12 g |
| Example 12 | 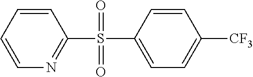 [SMILES: O=S(=O)(c1ccc(C(F)(F)F)cc1)c1ccccn1] | 0.15 g |
| Example 13 | 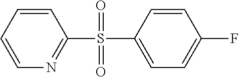 [SMILES: O=S(=O)(c1ccc(F)cc1)c1ccccn1] | 0.15 g |
| Example 14 | 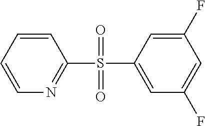 [SMILES: O=S(=O)(c1cc(F)cc(F)c1)c1ccccn1] | 0.12 g |
| Example 15 | 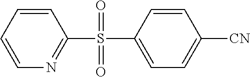 [SMILES: N#Cc1ccc(S(=O)(=O)c2ccccn2)cc1] | 0.10 g |
| Example 16 | 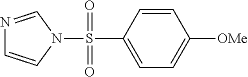 [SMILES: COc1ccc(S(=O)(=O)n2ccnc2)cc1] | 0.18 g |
| Example 17 | 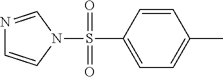 | 0.10 g |
| Example 18 | 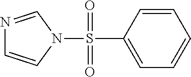 [SMILES: O=S(=O)(c1ccccc1)n1ccnc1] | 0.05 g |
| Example 19 | 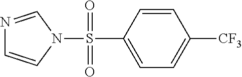 [SMILES: O=S(=O)(c1ccc(C(F)(F)F)cc1)n1ccnc1] | 0.12 g |
| Example 20 | 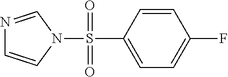 [SMILES: O=S(=O)(c1ccc(F)cc1)n1ccnc1] | 0.15 g |
| Example 21 | 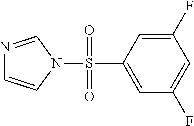 [SMILES: O=S(=O)(c1cc(F)cc(F)c1)n1ccnc1] | 0.15 g |
| Example 22 | 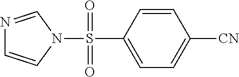 [SMILES: N#Cc1ccc(S(=O)(=O)n2ccnc2)cc1] | 0.15 g |
| Example 23 | 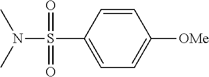 | 0.12 g |
| Example 24 | 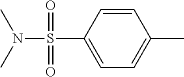 | 0.12 g |
| Example 25 | 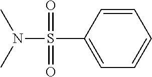 | 0.12 g |
| Example 26 | 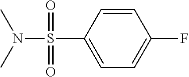 | 0.12 g |
| Example 27 | 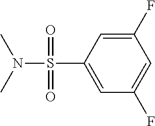 | 0.10 g |
| Example 28 | 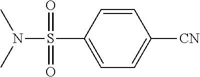 | 0.15 g |
| Example 29 | 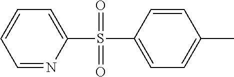 [SMILES: Cc1ccc(S(=O)(=O)c2ccccn2)cc1] | 0.10 g |
| Example 30 | 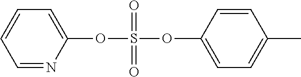 [SMILES: Cc1ccc(OS(=O)(=O)Oc2ccccn2)cc1] | 0.12 g |
| Example 31 | 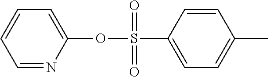 [SMILES: Cc1ccc(S(=O)(=O)Oc2ccccn2)cc1] | 0.12 g |
| Example 32 | 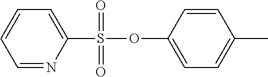 [SMILES: Cc1ccc(OS(=O)(=O)c2ccccn2)cc1] | 0.12 g |
| Example 33 | 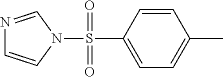 | 0.10 g |
| Example 34 | 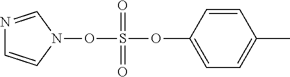 [SMILES: Cc1ccc(OS(=O)(=O)On2ccnc2)cc1] | 0.12 g |
| Example 35 | 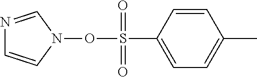 [SMILES: Cc1ccc(S(=O)(=O)On2ccnc2)cc1] | 0.12 g |
| Example 36 | 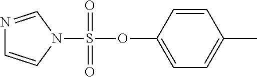 [SMILES: Cc1ccc(OS(=O)(=O)n2ccnc2)cc1] | 0.12 g |
| Example 37 | 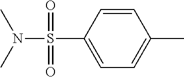 | 0.12 g |
| Example 38 | 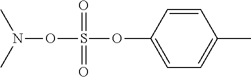 | 0.12 g |
| Example 39 | 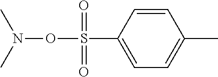 | 0.10 g |
| Example 40 | 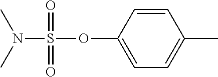 | 0.10 g |
| Example 41 | 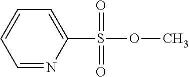 | 0.10 g |
| Example 42 | 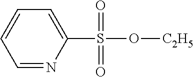 [SMILES: CCOS(=O)(=O)c1ccccn1] | 0.12 g |
| Example 43 | 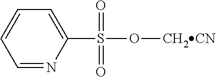 | 0.10 g |
| Example 44 | 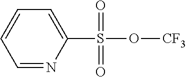 [SMILES: O=S(=O)(OC(F)(F)F)c1ccccn1] | 0.15 g |
| Example 45 | 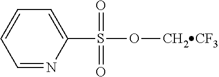 | 0.05 g |
| Example 46 | 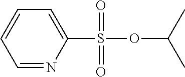 | 0.12 g |
| Example 47 | 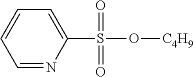 | 0.01 g |
| Example 48 | 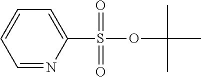 | 0.01 g |
| Example 49 | 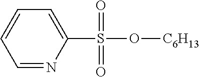 | 0.10 g |
| Example 50 | 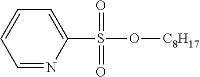 | 0.12 g |
| Example 51 | 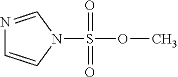 | 0.12 g |
| Example 52 | 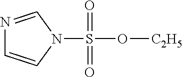 [SMILES: CCOS(=O)(=O)n1ccnc1] | 0.05 g |
| Example 53 | 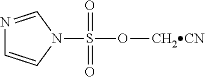 | 0.12 g |
| Example 54 | 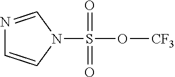 [SMILES: O=S(=O)(OC(F)(F)F)n1ccnc1] | 0.12 g |
| Example 55 | 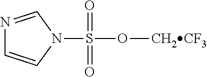 | 0.10 g |
| Example 56 | 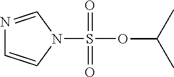 | 0.12 g |
| Example 57 | 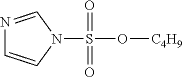 | 0.10 g |
| Example 58 | 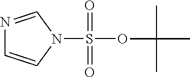 | 0.12 g |
| Example 59 | 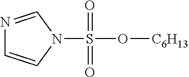 | 0.05 g |
| Example 60 | 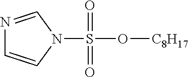 | 0.10 g |
| Comparative | none | none |
| Example 1 | | |
| Comparative Example 2 Comparative Example 3 | 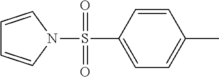 | 0.10 g 0.20 g |
| Comparative Example 4 Comparative Example 5 | 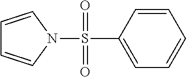 [SMILES: O=S(=O)(c1ccccc1)n1cccc1] | 0.10 g 0.20 g |

### Test of Battery Performance

### Measurement of Charge/Discharge Capacity and Measurement of Capacity Retention Ratio

First activation and cycle tests were performed on a Neware battery tester (model 5 V-50 mA). First, charge and discharge were performed once at 0.1 C at room temperature. Cycle test: 100 cycle tests with a charge of 0.5 C and a discharge of 5 C were conducted under the condition of 60° C.

Cycle retention ratio after 100 cycles [%]=(discharge capacity at the 100th cycle/discharge capacity at the 1st cycle)×100%

### Impedance Measurement

The lithium ion secondary battery was kept at room temperature, charged once at 0.5 C, and left at room temperature for 1 hour. Then the initial impedance value of the battery was tested by an electrochemical workstation with a frequency range of 500 MHz-50 mHz. 100 charge-discharge cycles were carried out at room temperature, and then charge test at 0.5 C was carried out once. After standing at room temperature for 1 hour, the final impedance value of the battery was tested by the electrochemical workstation with a frequency range of 500 MHz-50 mHz.

$$Impedance growth rate [ \% ] = ( final impedance value - initial impedance value ) \text{} / initial impedance value \times 100 \%$$

The experimental results are shown in Table 2 below.

TABLE 2

| | | Post-cycle | |
|---|---|---|---|
| | Additive | impedance | Cycle |
| | amount of | growth | retention |
| | electrolyte | rate | ratio |
| Example 1 | 0.01 g | 139% | 77% |
| Example 2 | 0.05 g | 135% | 79% |
| Example 3 | 0.10 g | 127% | 82% |
| Example 4 | 0.12 g | 111% | 84% |
| Example 5 | 0.15 g | 118% | 88% |
| Example 6 | 0.18 g | 125% | 84% |
| Example 7 | 0.20 g | 135% | 79% |
| Example 8 | 0.25 g | 134% | 75% |
| Example 9 | 0.10 g | 124% | 83% |
| Example 10 | 0.10 g | 122% | 82% |
| Example 11 | 0.12 g | 114% | 74% |
| Example 12 | 0.15 g | 115% | 81% |
| Example 13 | 0.15 g | 115% | 82% |
| Example 14 | 0.12 g | 114% | 74% |
| Example 15 | 0.10 g | 117% | 83% |
| Example 16 | 0.18 g | 152% | 82% |
| Example 17 | 0.10 g | 120% | 80% |
| Example 18 | 0.05 g | 123% | 84% |
| Example 19 | 0.12 g | 113% | 84% |
| Example 20 | 0.15 g | 123% | 82% |
| Example 21 | 0.15 g | 119% | 85% |
| Example 22 | 0.15 g | 108% | 84% |
| Example 23 | 0.12 g | 123% | 84% |
| Example 24 | 0.12 g | 120% | 84% |
| Example 25 | 0.12 g | 122% | 79% |
| Example 26 | 0.12 g | 124% | 80% |
| Example 27 | 0.10 g | 131% | 79% |
| Example 28 | 0.15 g | 107% | 88% |
| Example 29 | 0.10 g | 122% | 82% |
| Example 30 | 0.12 g | 118% | 80% |
| Example 31 | 0.12 g | 119% | 85% |
| Example 32 | 0.12 g | 118% | 82% |
| Example 33 | 0.10 g | 120% | 80% |
| Example 34 | 0.12 g | 118% | 82% |
| Example 35 | 0.12 g | 120% | 84% |
| Example 36 | 0.12 g | 120% | 83% |
| Example 37 | 0.12 g | 120% | 84% |
| Example 38 | 0.12 g | 121% | 79% |
| Example 39 | 0.10 g | 133% | 82% |
| Example 40 | 0.10 g | 130% | 80% |
| Example 41 | 0.10 g | 126% | 80% |
| Example 42 | 0.12 g | 123% | 83% |
| Example 43 | 0.10 g | 135% | 81% |
| Example 44 | 0.15 g | 111% | 80% |
| Example 45 | 0.05 g | 133% | 79% |
| Example 46 | 0.12 g | 118% | 81% |
| Example 47 | 0.01 g | 133% | 79% |
| Example 48 | 0.01 g | 134% | 81% |
| Example 49 | 0.10 g | 128% | 85% |
| Example 50 | 0.12 g | 113% | 83% |
| Example 51 | 0.12 g | 115% | 82% |
| Example 52 | 0.05 g | 132% | 83% |
| Example 53 | 0.12 g | 113% | 83% |
| Example 54 | 0.12 g | 122% | 85% |
| Example 55 | 0.10 g | 135% | 85% |
| Example 56 | 0.12 g | 115% | 84% |
| Example 57 | 0.10 g | 129% | 81% |
| Example 58 | 0.12 g | 122% | 84% |
| Example 59 | 0.05 g | 135% | 82% |
| Example 60 | 0.10 g | 127% | 83% |
| Comparative | none | 139% | 74% |
| Example 1 | | | |
| Comparative | 0.10 g | 137% | 73% |
| Example 2 | | | |
| Comparative | 0.20 g | 138% | 75% |
| Example 3 | | | |
| Comparative | 0.10 g | 145% | 74% |
| Example 4 | | | |
| Comparative | 0.20 g | 137% | 72% |
| Example 5 | | | |

It can be determined from the data shown in Table 2 that compared with Comparative Example 1 without the electrolyte additive of the present disclosure, Examples 1-60 of the present disclosure all achieved a significantly increased cycle retention ratio, and Example 28 even increased the cycle retention ratio to astonishing 88%. Although the cycle retention ratio of the battery comprising the electrolyte additive of Examples 11 and 14 was the same as that of Comparative Example 1, the post-cycle impedance growth rate was significantly reduced from 139% (Comparative Example 1) to 114% (Example 1), and thus the electrical performance of the battery was improved. Although Example 1 and Example 16 were slightly inferior to Comparative Example 1 in terms of the post-cycle impedance growth rate, significant growth was achieved in terms of the cycle retention ratio. It is well known in the art that the impedance of a battery may grow uncontrollably upon addition of any electrolyte additive. For long cycle batteries, which require a higher cycle life, a slightly higher impedance is acceptable if it has little effect on the cycle retention ratio or beneficially increases the cycle retention ratio. Both Example 1 and Example 16 of the present disclosure achieved an excellent cycle retention ratio with lower increase in post-cycle impedance growth rate. In addition, the post-cycle impedance growth rates of the other examples of Examples 1-60 described above all exhibited significant reduction.

By comparison of Examples 1-60 of the present disclosure with Comparative Examples 2-5 containing other electrolyte additives, although both of the compounds of Comparative Examples 2-5 contain N and S atoms, the nitrogen element in the compound has already bonded to the sulfur element, there are no remaining electrons on nitrogen atom similar to the related compounds of the present disclosure, that is to say, none of the compounds of comparative examples 2-5 has a basic property. When such a non-alkaline compound is used as an electrolyte additive, an electrode protection film cannot be effectively formed. Therefore, it is impossible to achieve a reduced post-cycle impedance growth rate and an excellent cycle retention ratio.

It should be understood that various changes and modifications to the embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. An electrolyte additive, comprising a compound of formula (1):
R₁-L-R₂ (1)
wherein
R₁ is selected from a substituted or unsubstituted five-membered heterocyclic group, a substituted or unsubstituted six-membered heterocyclic group, or a substituted or unsubstituted amino group;
L is a sulfone group, a sulfate ester group, or a sulfonate ester group;
R₂ is selected from a substituted or unsubstituted C₁₋₁₈ alkyl group, a substituted or unsubstituted C₁₋₁₈ alkoxy group, a substituted or unsubstituted conjugated five-membered carbocyclic group or heterocyclic group, a substituted or unsubstituted conjugated six-membered carbocyclic group or heterocyclic group, a di(C₁₋₆ alkyl)amino group, a di(C₆₋₁₂ aryl)amino group, a tri(C₁₋₆ alkyl)silyl group, a substituted or unsubstituted C₃₋₆ alkenyl group, a cyano group, a substituted or unsubstituted C₃₋₆ alkynyl group, an ether group, or a substituted or unsubstituted ester group.

2. The electrolyte additive of claim 1, wherein in the compound of formula (1), R₁ is a substituted or unsubstituted pyridyl group, a substituted or unsubstituted pyrazinyl group, a substituted or unsubstituted imidazolyl group, or a (C₁₋₃ alkyl) substituted or unsubstituted amino group.

3. The electrolyte additive of claim 2, wherein in the compound of formula (1), R₁ is a pyridyl group, a pyrazinyl group, an imidazolyl group, an amino group, a dimethylamino group, or a diethylamino group.

4. The electrolyte additive of claim 1, wherein in the compound of formula (1), R₂ is selected from a substituted or unsubstituted C₁₋₈ alkyl group, a substituted or unsubstituted C₁₋₈ alkoxy group, a substituted or unsubstituted conjugated six-membered carbocyclic group or heterocyclic group, a substituted or unsubstituted —COO(C₁₋₆), di(C₁₋₃ alkyl)amino group or di(C₆₋₁₂ aryl)amino group, a cyano group, or an ether group.

5. The electrolyte additive of claim 4, wherein in the compound of formula (1), R₂ is selected from a C₁₋₃ alkyl substituted or unsubstituted conjugated six-membered carbocyclic group or heterocyclic group, a C₁₋₃ alkoxy substituted conjugated six-membered carbocyclic group or heterocyclic group, a fluoro substituted conjugated six-membered carbocyclic group or heterocyclic group, a fluoromethyl substituted conjugated six-membered carbocyclic group or heterocyclic group, a difluoromethyl substituted conjugated six-membered carbocyclic group or heterocyclic group, a trifluoromethyl substituted conjugated six-membered carbocyclic group or heterocyclic group, a cyano substituted conjugated six-membered carbocyclic group or heterocyclic group, a cyano substituted or unsubstituted C₁₋₈ alkyl group, a cyano substituted or unsubstituted C₁₋₈ alkoxy group, a di(C₁₋₃ alkyl)amino group or di(C₆₋₁₂ aryl)amino group, or a cyano group.

6. The electrolyte additive of claim 5, wherein in the compound of formula (1), R₂ is selected from phenyl, p-methylphenyl, p-ethylphenyl, m-methylphenyl, m-ethylphenyl, 3,5-dimethylphenyl, 3,5-diethylphenyl, o-methylphenyl, 2,6-dimethylphenyl, imidazolyl, pyridyl, pyrimidinyl, pyridazinyl, pyrazinyl, —CH₃, —OCH₃, —C₂H₅, —OC₂H₅, —C₃H₇, —OC₃H₇, n-butyl, isobutyl, t-butyl, —C₆H₁₃, —C₁₈H₃₇, —COOCH₃, —COOC₂H₅, —CN, —CH₂CN, dimethylamino, diethylamino, dipropylamino, trimethylsilyl, triethylsilyl, triisopropylsilyl, vinyl, or ethynyl.

7. The electrolyte additive of claim 1, wherein the compound of formula (1) is one of:

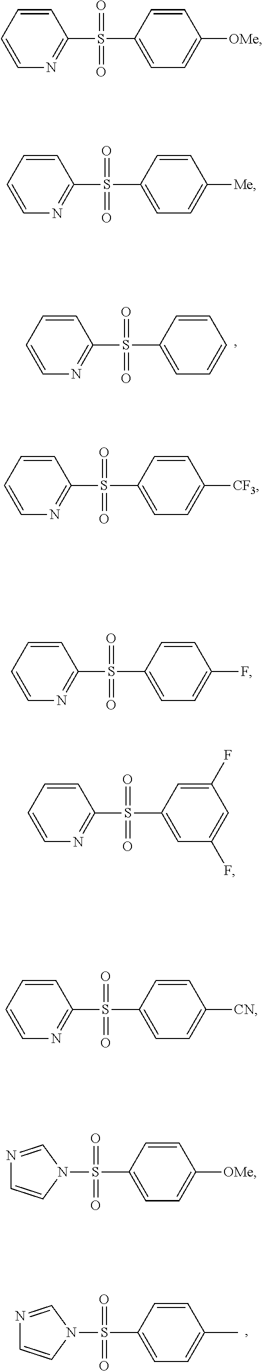

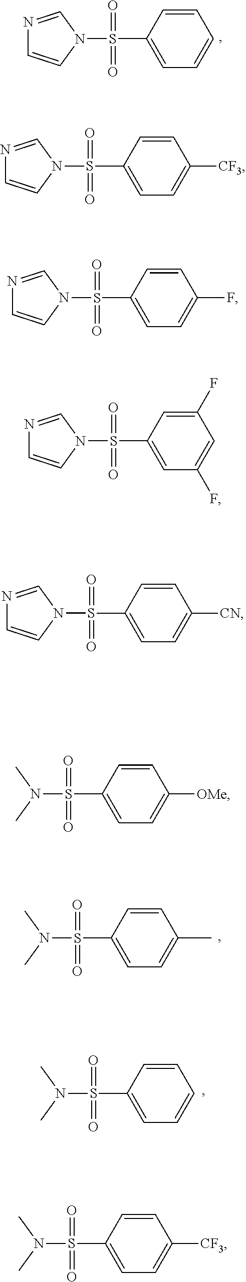

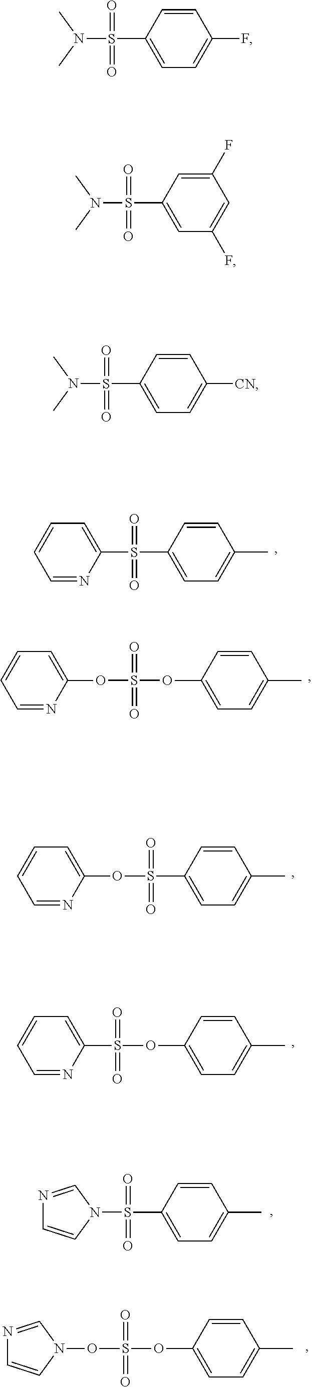

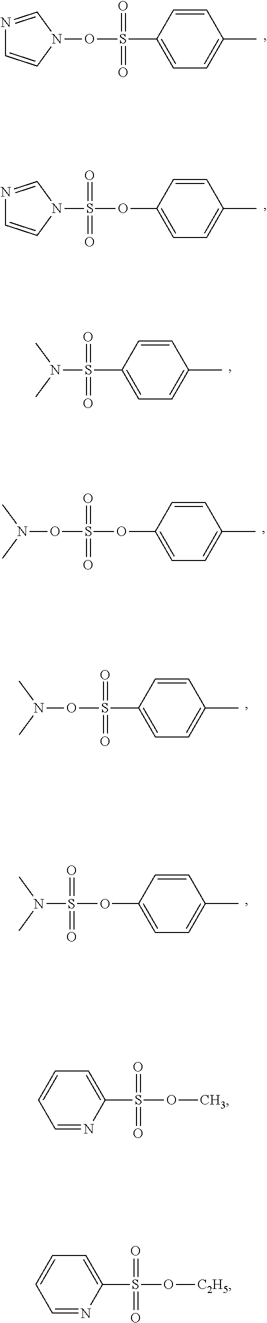

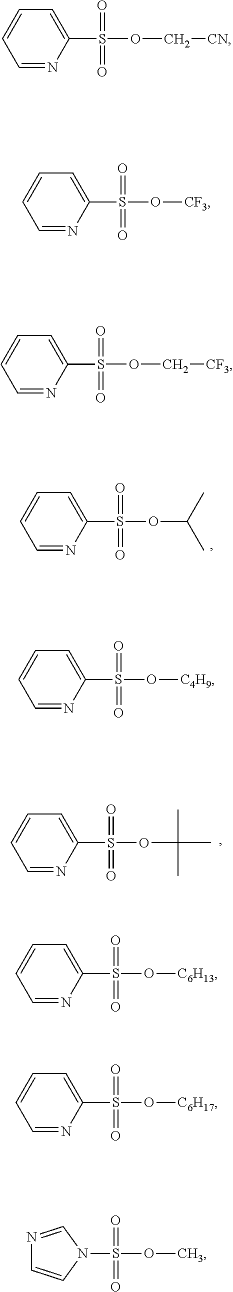

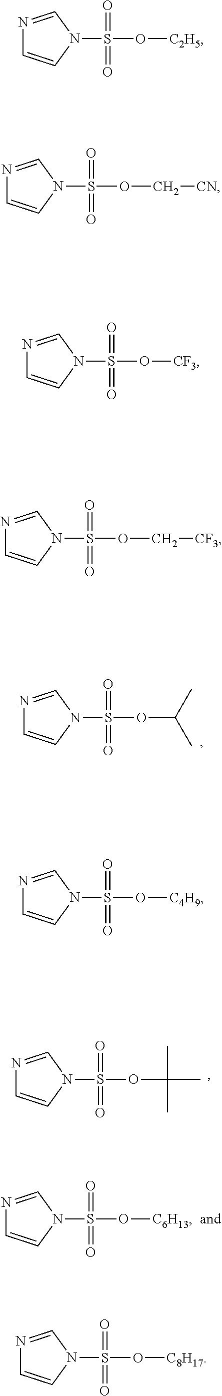

8. An electrolyte, comprising an organic solvent, a lithium salt, and the electrolyte additive of claim 1.

9. The electrolyte of claim 8, wherein an amount of the electrolyte additive is in a range of 0.01 parts by weight to 0.25 parts by weight based on 100 parts by weight of a total weight of the organic solvent and the lithium salt.

10. The electrolyte of claim 8, wherein the lithium salt is one or more of LiCl, LiBr, LiPF₆, LiBF₄, LiAsF₆, LiClO₄, LiB(C₆H₅)₄, LiCH₃SO₃, LiCF₃SO₃, LiN(SO₂F)₂, LiN(SO₂CF₃)₂, LiC(SO₂CF₃)₃, LiAlCl₄, and LiSiF₆.

11. The electrolyte of claim 8, wherein the organic solvent is one or more of ethylene carbonate, propylene carbonate, butylene carbonate, fluorovinyl carbonate, diethyl carbonate, dipropyl carbonate, methyl ethyl carbonate, vinyl carbonate, dimethyl carbonate.

12. A lithium ion secondary battery, comprising:
a positive electrode sheet,
a negative electrode sheet,
a separator,
the electrolyte of claim 8.
